(19) United States
(12) Patent Application Publication
(10) Pub. No.: US 2013/0277410 A1
(43) Pub. Date: Oct. 24, 2013

(54) SAFETY LOCKOUT FOR SURGICAL STAPLER

(71) Applicant: CARDICA, INC., Redwood City, CA (US)

(72) Inventors: Anthony J. Fernandez, San Carlos, CA (US); Bryan D. Knodel, Flagstaff, AZ (US); Jinhoon Park, Mountain View, CA (US)

(73) Assignee: Cardica, Inc., Redwood City, CA (US)

(21) Appl. No.: 13/859,808

(22) Filed: Apr. 10, 2013

Related U.S. Application Data
(60) Provisional application No. 61/635,116, filed on Apr. 18, 2012.

(51) Int. Cl. A61B 17/068

(52) U.S. Cl. CPC A61B 17/068
USPC 227/175.2; 227/180.1

(57) ABSTRACT

A surgical stapling and cutting device is constructed with a knife safety lockout element to prevent a knife element from cutting tissue if staples cannot be applied first to the tissue prior to being cut. The surgical stapling and cutting device comprises of a staple holder for holding a staple cartridge, an anvil movably coupled to the staple holder for opposing and deforming staples, a knife safety lockout element for blocking a knife element from being deployed, and a safety spring element for activating or biasing the knife safety lockout element into an active state to block the knife element from being deployed.

### CROSS-REFERENCE TO RELATED APPLICATION

This application claims priority of U.S. Provisional Patent Application Ser. No. 61/635,116, Attorney Docket No. 349, filed on Apr. 18, 2012, which is hereby incorporated by reference in its entirety.

### FIELD OF THE INVENTION

The invention is generally related to surgical stapling devices.

### BACKGROUND

A linear cutter is a surgical instrument that staples and cuts tissue, such as gastrointestinal tissue or other tissues of a patient. The linear cutter staples and cuts the target tissue such that the cut portions of the transected tissue are hemostatic. A typical linear cutter holds a disposable single-use staple cartridge with several rows of staples held within it, and it usually includes an anvil to oppose the staples as they are deployed from the staple cartridge. During a surgical operation, such as a minimally invasive procedure, the surgeon inserts the linear cutter through an opening in the body, orients the end of the linear cutter around the target tissue to be transected, and compresses the anvil and cartridge together to clamp that tissue. Then, a row or rows of staples are deployed on either side of the transection line, and a blade is advanced along the transection line to divide or cut the tissue. Since the divided or cut portions of the tissue were stapled, they are hemostatic after the cut.

During actuation of a linear cutter, the cartridge typically fires all of the staples that it holds. In order to deploy more staples, the linear cutter must be moved away from the surgical site and removed from the patient, after which the old cartridge is exchanged for a new cartridge. The linear cutter is then reinserted into the patient for additional procedures.

### SUMMARY

In accordance with one embodiment, a surgical stapling and cutting device is constructed with a knife safety lockout element to prevent a knife element from cutting tissue if staples cannot be applied first to the tissue prior to being cut. The surgical stapling and cutting device comprises of a staple holder for holding a staple cartridge, an anvil movably coupled to the staple holder for opposing and deforming staples, a knife safety lockout element for blocking a knife element from being deployed, and a safety spring element for activating or biasing the knife safety lockout element into an active state to block the knife element from being deployed.

### BRIEF DESCRIPTION OF THE DRAWINGS

FIG. 1 illustrates examples of linear cutters and stapling systems.

FIG. 2A through FIG. 2C illustrate an example of a jaw assembly of a linear cutter and stapling system.

FIG. 3A illustrates an opened jaw assembly of a linear cutter and stapling system.

FIG. 3B illustrates a close-up view of an opened jaw assembly of a linear cutter and stapling system.

FIG. 4A through FIG. 4C illustrate a safety lock-out mechanism for a surgical knife of a linear cutter and stapling system.

FIG. 5A through FIG. 5D illustrate a safety lock-out mechanism for a surgical knife of a linear cutter and stapling system.

FIG. 6A through FIG. 6D illustrate a safety lock-out mechanism for a surgical knife of a linear cutter and stapling system being deactivated by a staple cartridge.

FIG. 7A and FIG. 7B illustrate a safety lock-out mechanism being deactivated.

### DETAILED DESCRIPTION

In the following detailed description, specific details are set forth in order to provide a thorough understanding of the present invention. However, it will be understood by those skilled in the art that the present invention may be practiced without these specific details. Alternatively, some of the well-known parts, component, hardware, methods of operations, and procedures may not be described in detail or elaborated so as to avoid obscuring the present invention; but, nevertheless, they are within the spirit and scope of the present invention.

A linear cutter is a surgical instrument that staples and cuts tissue, such as gastrointestinal tissue or other tissues of a patient. The linear cutter staples and cuts the target tissue such that the cut portions of the transected tissue are hemostatic. A typical linear cutter holds a disposable single-use staple cartridge with several rows of staples held within it, and it usually includes an anvil to oppose the staples as they are deployed from the staple cartridge. During a surgical operation, such as a minimally invasive procedure, the surgeon inserts the linear cutter through an opening in the body, orients the end of the linear cutter around the target tissue to be transected, maneuvers the jaw of the linear cutter until the target tissue is in-between the open jaw, and compresses the anvil and staple cartridge together to clamp that portion of the tissue to be cut. Then, a row or rows of staples are deployed on either side of the transection line, and a blade is advanced along the transection line to divide or cut the tissue. Since the divided or cut portions of the tissue were stapled, they are hemostatic after the cut.

During actuation of a linear cutter, the staple cartridge typically fires all of the staples that it holds. In order to deploy more staples, the linear cutter must be moved away from the surgical site and removed from the patient, after which the old cartridge is exchanged for a new cartridge. The linear cutter is then reinserted into the patient with a new staple cartridge for additional stapling and cutting as required by the surgical procedure.

As can be appreciated from the above discussion, stapling a target tissue to be transected prior to cutting the tissue avoids internal bleeding, which can facilitate the surgical procedure as the surgeon would not have deal with bleeding tissue. In addition, sealing or stapling the tissue prior to cutting may reduce trauma to the tissue as well as possibly expedite the healing and recovery process to the transected tissue, which may lead to long term positive outcome for the surgical operation. As such, the knife should not be deployed when a staple cartridge is not properly loaded in the linear cutter for staple deployment, become dislodged, missing, spent, or empty. To address this issue, Cardica, Inc. (“Cardica”) of Redwood City, California, has developed a safety lockout to prevent deployment of the knife when the staple cartridge is dislodged, missing, spent, or empty.

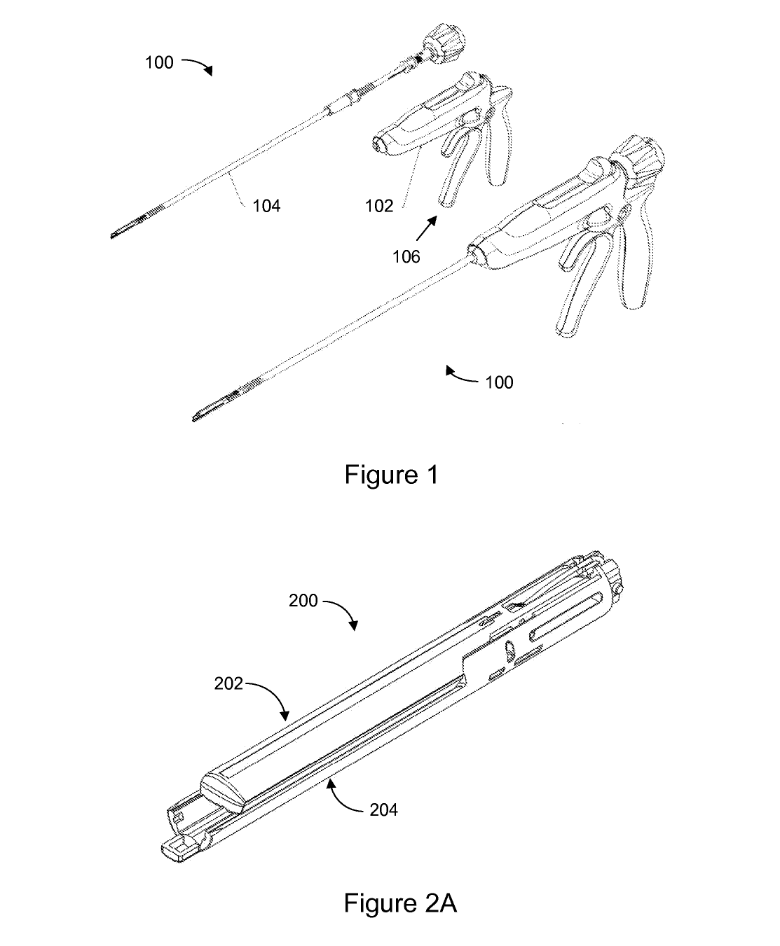

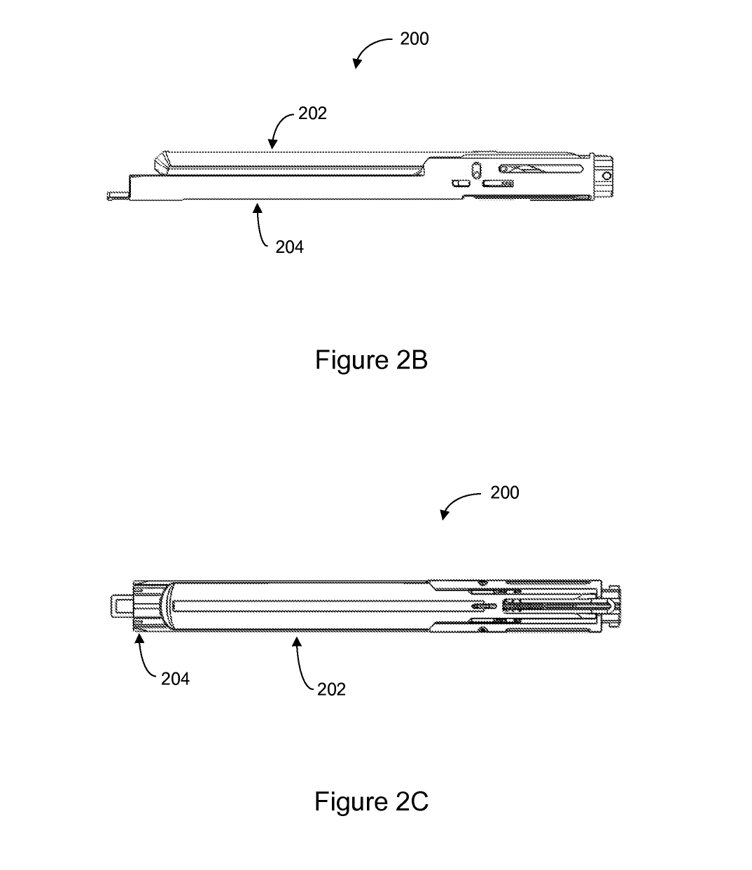

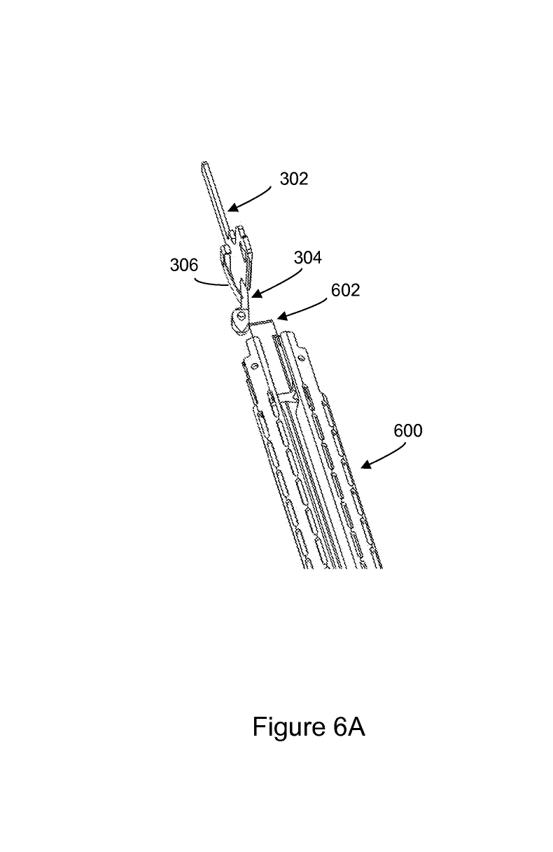

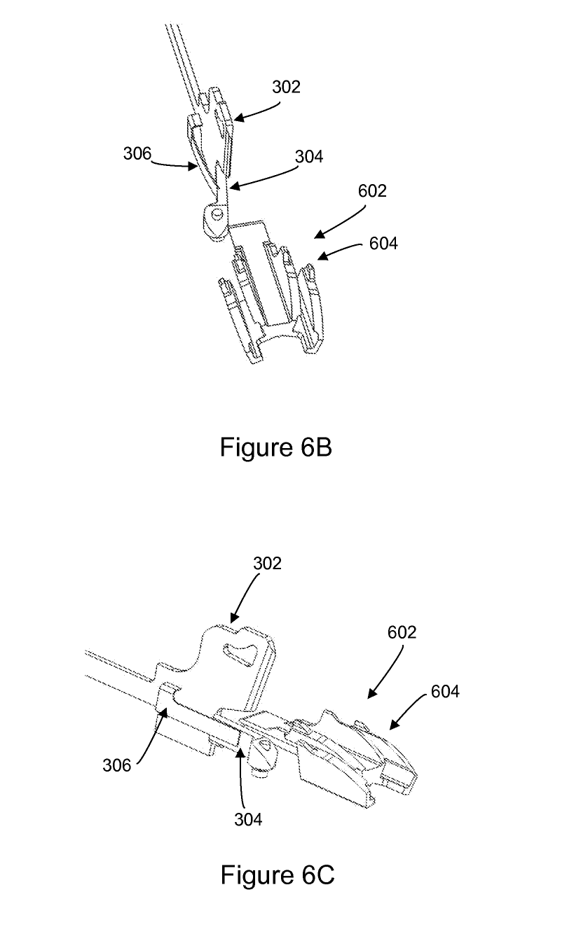

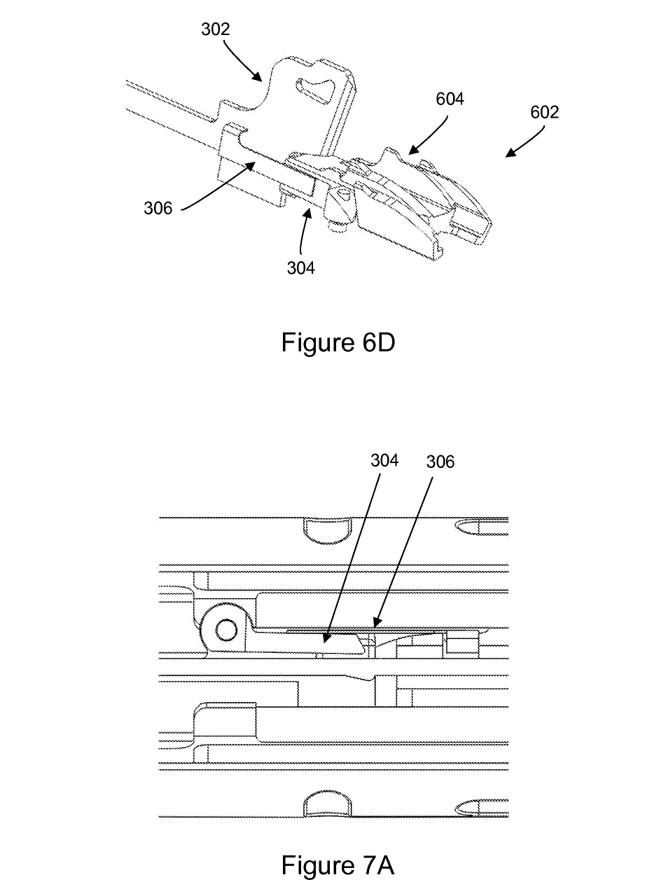

FIG. 1 illustrates an example of linear cutter 100, such as the MicroCutter XChange™ 30 that is designed and manufactured by Cardica, Inc. (“Cardica”) of Redwood City, Calif. Typically, the linear cutter 100 includes a handle assembly 102 and a shaft assembly 104. The handle assembly 102 usually includes various trigger and drive mechanisms for deploying staples and knife to staple and cut tissues. The shaft assembly 104 usually includes mechanisms and components for delivering and deploying the staples and knife at the point of action to staple and cut tissues. For example, the shaft assembly 104 typically includes a jaw assembly 200, illustrated in FIG. 2A through FIG. 2C, at the distal portion of the shaft assembly 104 that acts directly on the tissue to be stapled and cut. The jaw assembly 200 typically includes an anvil 202 that is movably coupled to a staple channel or staple holder 204. The staple channel 204 typically holds a staple cartridge (not shown) that stores and deploys staples. The staples are deployed by one or more wedges 604 in a wedge assembly 602 (shown in FIG. 6A through FIG. 6D). The anvil 202 acts in concert with the staple holder 204, with a loaded staple cartridge in the staple holder, the linear cutter clamps the target tissue, and then staples and cuts the target tissue upon activation of the trigger element and various mechanisms in the handle assembly 102.

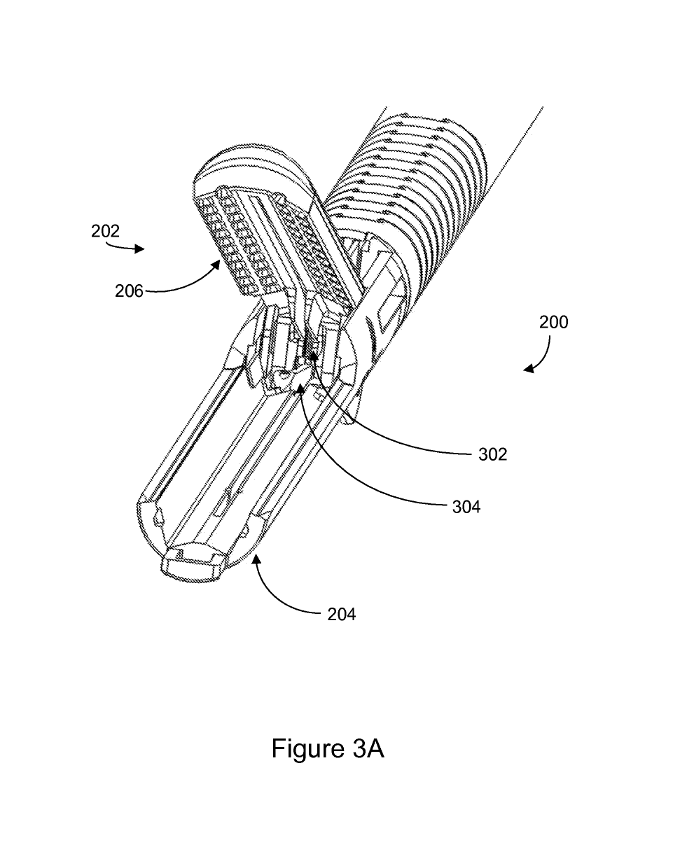

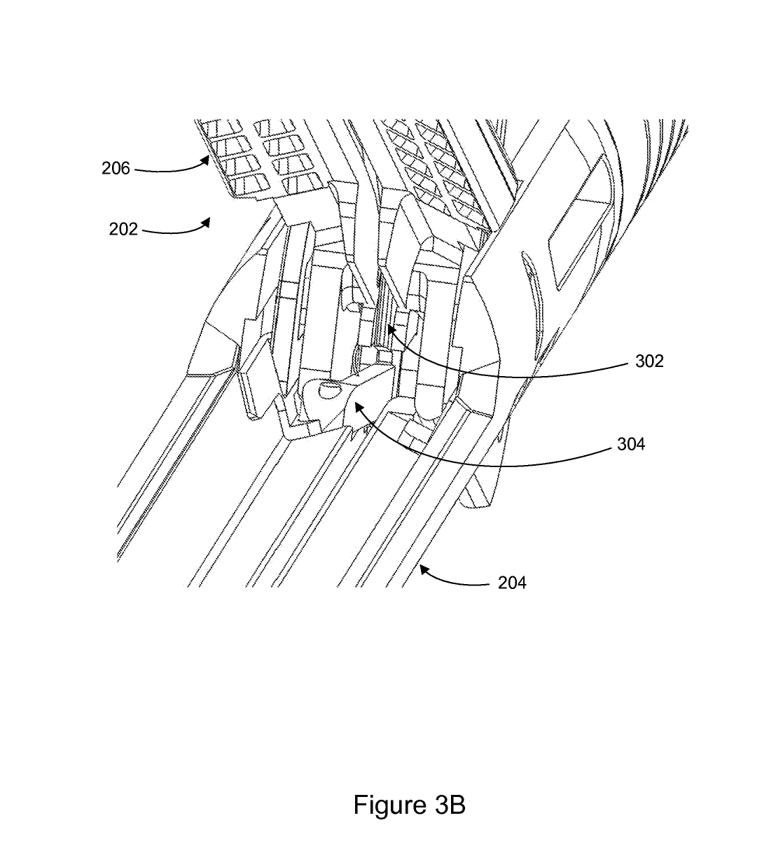

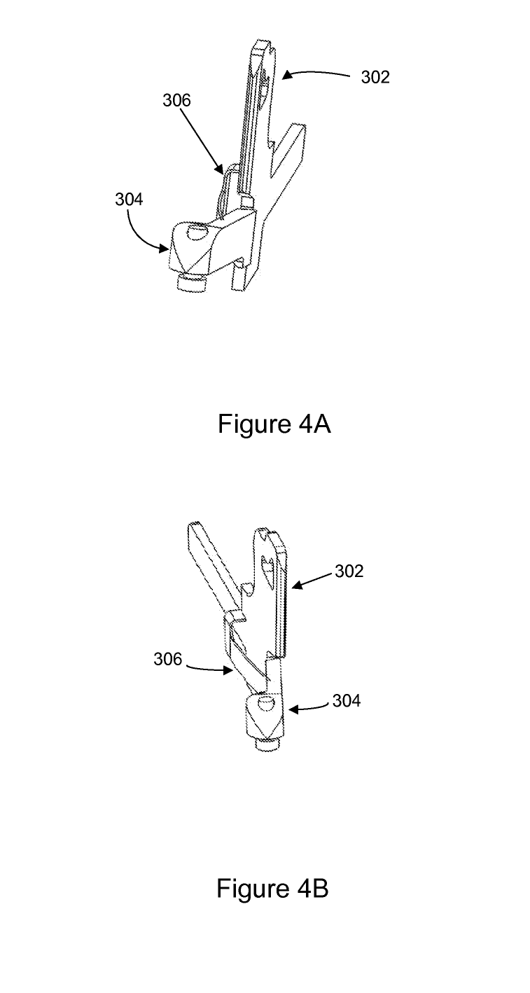

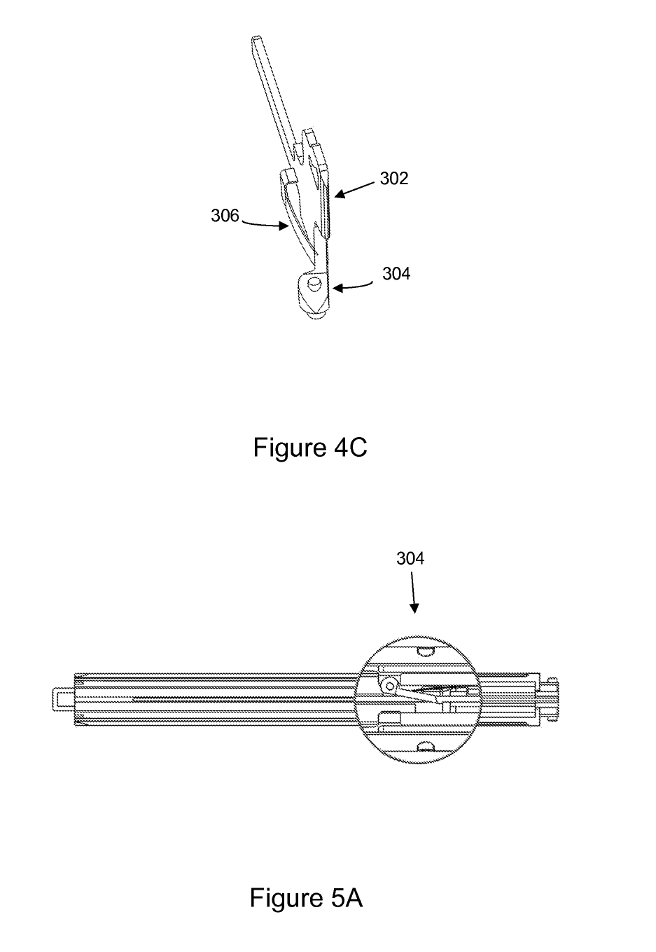

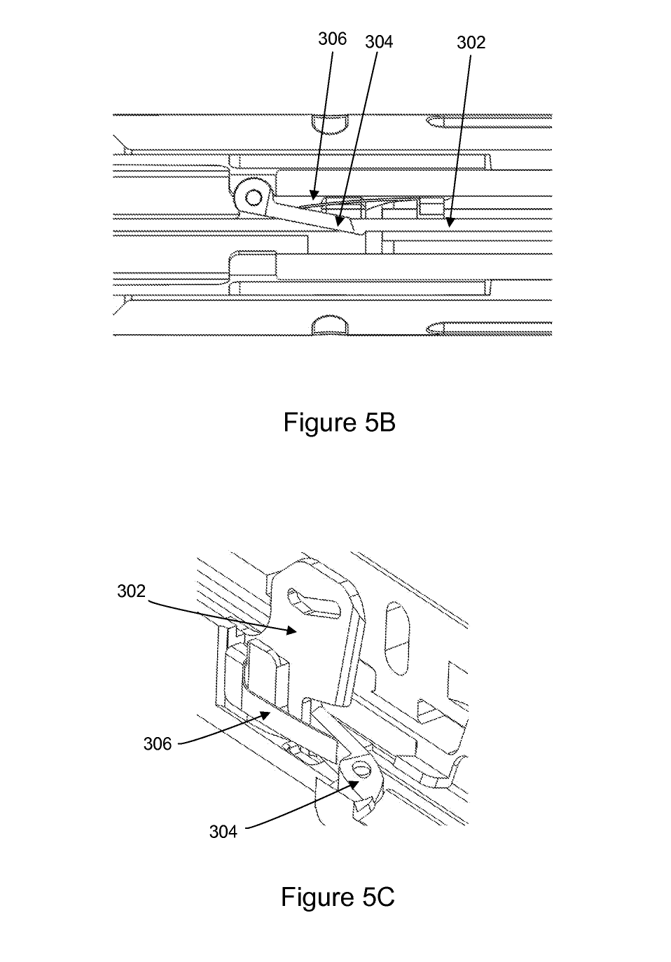

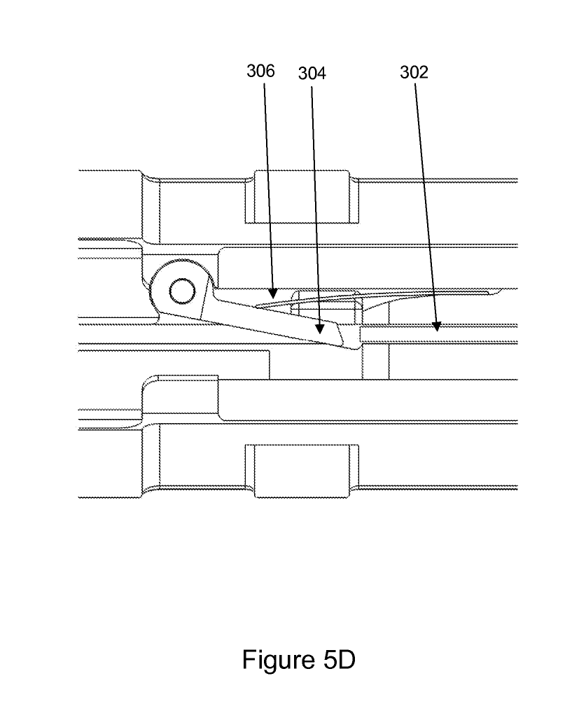

FIG. 3A and FIG. 3B illustrate close-up views of the jaw assembly 200 in an open-jaw orientation. In this open-jaw orientation, the knife 302 for cutting tissue and the safety lockout element 304 are visible inside the open-jaw of the stable holder 204. FIG. 4A through FIG. 4C provide illustrations of isolated elements of the knife 302 and safety lockout elements (e.g., knife safety lockout 304 and safety spring element 306). As can be appreciated from these figures, the knife safety lockout element 304 may be biased by the safety spring element 306 to an orientation or position that is substantially in front of the knife 302. In this manner, the knife safety lockout element 304 is directly in the path of travel for the knife 302, which prevents it from being deployed to cut and transect tissue. The knife safety lockout element 304 may be biased substantially in a horizontal direction or plane to prevent the knife 302 from being deployed. Similarly, the knife safety lockout element 304 may be biased substantially in a diagonal direction or plane. Alternatively, the knife safety lockout element 304 may be biased substantially in a vertical direction or plane. As one example, the biasing of the knife safety lockout element 304 by the spring element 306 may be analogous to that of a gate or door being swing substantially horizontally to place the knife lockout element 304 to an active state. Alternatively, biasing the knife safety lockout element 304 in an opposite direction may place the lockout element 304 in a neutral state. In one example, the spring element 306 may be a sheet spring element, a leaf spring element, or a rod spring element. In another example, the spring element 306 may be a coil spring element or a torsional spring element. To be discussed further, the knife safety lockout may be biased away from the front of the knife 302 and out of its path of travel during operation. FIG. 5A illustrates a close-up top view of the knife safety lockout element 304. FIG. 5B illustrates a further close-up top view of the knife safety lockout element 304 in relation to the knife 302 and safety spring element 306 to provide a better understanding of their orientations and interactions. FIG. 5C illustrates an isometric view or perspective view of the surgical knife element 302, the knife safety lockout 304, and the safety spring element 306. FIG. 5D illustrates a even further close-up top view of the knife safety lockout element 304 in relation to the knife 302 and safety spring element 306 to provide a clearer illustration of their orientations and interactions. As clearly shown in these figures, the safety spring element 306 provides the necessary force to bias or push the safety lockout element 304 in front of the knife element 302 blocking its path of travel such that the knife 302 cannot be deployed. As can be appreciated from the figures, the safety lockout 304 is pushed or biased in front of the knife 302 when the staple cartridge is missing or dislodged. To be discussed further, the safety lockout 304 is pushed or biased in front of the knife 302 after the staples held in the staple cartridge are deployed and the knife 302 is reset to its initial or ready position. To be more clear, once some (one or more) of the staples have been deployed, resetting the knife to its initial or ready position will activate the safety lockout 304 and prevent the knife 302 from moving or advancing to be deployed and cut tissue. As such, the knife safety lockout element 304 prevents the knife 302 from being deployed without having staples being deployed first and stapling the tissue that will be cut by the knife 302.

FIG. 6A through FIG. 6D illustrate engagement or placement of a staple cartridge 600 to the staple holder 204 which will deactivate the knife safety lockout 304. More specifically, the wedge assembly 602 may deactivate the knife safety lockout 304. For example, the wedge assembly 602 will push against the knife safety lockout element 304 and the safety spring element 306 to overcome the spring force of the safety spring element 306 to place the knife safety lockout element 304 in a neutral position away from or out of the path of travel of the surgical knife element 302. In another example a different element or assembly of the staple cartridge 600 may deactivate the knife safety lockout element 304. Typically, the wedge assembly 602 and wedges 604 are used to deploy the staples in the staple cartridge 600 loaded into the staple holder 204 of the jaw assembly 200 at the distal portion of the shaft assembly 104 of the linear cutter 100.

During operation of the linear cutter 100, a surgeon operates the linear cutter 100 by activating the trigger element 106 of the handle assembly 102 to place the linear cutter 100 in a “trocar mode”. In the trocar mode, the end effector (e.g., the jaw portion of the shaft assembly 104) is at its smallest cross-sectional diameter for insertion through a substantially small opening. For example, the surgeon inserts the shaft of the linear cutter 100, through a small cut- out opening, a port, or a trocar, into a cavity of a patient. The surgeon then maneuvers the distal portion or end effector of the linear cutter 100, and places the jaw of the linear cutter 100 near the target tissue for the stapling and cutting of the tissue. The surgeon opens the jaw and clamps the target tissue between the anvil member 202 and the cartridge/staple holder 204 of the jaw assembly. The surgeon then further operates the trigger element 106, which drives the gears, pulleys, other components, etc. in the handle assembly 102 and shaft assembly 104 to advance the wedge assembly 602, wedge elements 604, and surgical knife element 302 to staple and cut the target tissue. As should be noted, the wedge assembly 602, more specifically the respective wedge elements 604, advances against each of the staples for deployment. For example, a wedge element 604 advances against a staple and pushes it to an upwardly arch motion toward a staple forming pocket 206 in the anvil 202, while frangibly separating the staple. The anvil 202 opposes and deforms the staple in the staple forming pocket 206.

During staple deployment and cutting of target tissue, the knife element 302 advances behind the wedge assembly 602 as one or more wedges 604 deploys the staples in the staple cartridge and the knife element 302 cuts the tissue along a transection line after the tissue is stapled on either or both sides of the transection line. As the staple cartridge is loaded into the staple holder 204, the wedge assembly 602 deactivates the knife safety lockout element 304 and places it in a neutral position. As both the wedge assembly 602 and knife element 302 are deployed in the stapling and cutting operation, the knife safety lockout 304 continues to be deactivated in a neutral position; first by the wedge assembly 602 and then by the body of the knife element 302. The knife safety lockout element continues to be deactivated in its neutral position until the knife element 302 is reset back to its initial ready position. The wedge assembly 604, on the other hand, remains in its deployed position or state. The wedge assembly 604 does not get reset; regardless if it has been partially or fully deployed. Instead, the staple cartridge with the partially or fully deployed or spent (empty) condition is replaced with a new or fully loaded staple cartridge. The new cartridge is loaded onto or into the staple holder 204. The loading of the new cartridge deactivates the knife safety lockout element 304 and places it in a neutral position. Once again, the linear cutter 100 is ready to be fired to deploy staples and cut tissue.

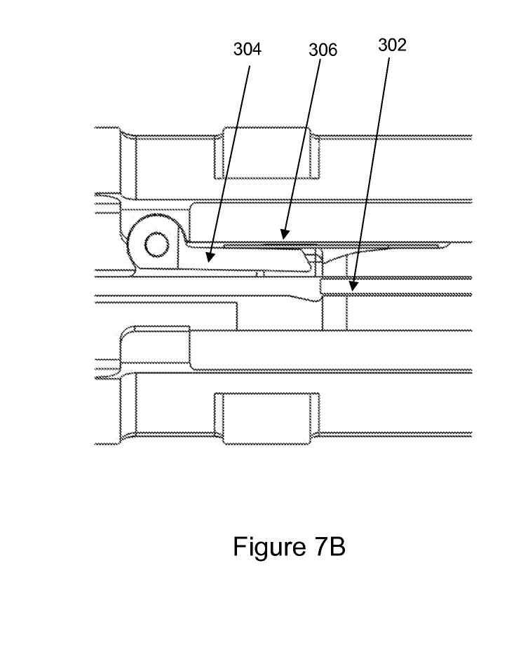

FIG. 7A and FIG. 7B illustrate the side-views of a new cartridge having been loaded into the staple holder 204, which deactivates the knife safety lockout element 304 and places in a neutral position away from the front of the surgical knife element 302 and out of it path of travel for cutting tissue as it is advanced following the wedge assembly 602.

While the invention has been described in detail, it will be apparent to one skilled in the art that various changes and modifications can be made and equivalents employed, without departing from the scope of the present invention. It is to be understood that the invention is not limited to the details of construction, the arrangements of components, and/or the method set forth in the above description or illustrated in the drawings. Statements in the abstract of this document, and any summary statements in this document, are merely exemplary; they are not, and cannot be interpreted as, limiting the scope of the claims. Further, the figures are merely exemplary and not limiting. Words such as “upper,” “lower,” “upward,” “downward”, “front”, “back”, “next to”, and the like are intended for the convenience of the reader and refer to the orientation and motion of parts on the printed page; they do not in any way limit the scope or application of the invention. Topical headings and subheadings, if provided, are for the convenience of the reader only. They should not and cannot be construed to have any substantive significance, meaning or interpretation, and should not and cannot be deemed to indicate that all of the information relating to any particular topic is to be found under or limited to any particular heading or subheading. Therefore, the invention is not to be restricted or limited. The present invention is intended to cover alternatives, modifications, and equivalents that may fall within the spirit and scope of the present invention as defined by the claims.

What is claimed is:

1. A surgical stapling and cutting device, comprising:
a staple holder for holding a staple cartridge;
an anvil movably coupled to the staple holder for opposing and deforming staples as staples are deployed from the staple cartridge;
a knife safety lockout element for blocking a knife element from being deployed; and
a safety spring element for activating or biasing the knife safety lockout element into an active state to block the knife element from being deployed.

2. The device of claim 1, wherein the knife safety lockout element is in the active state when the knife element is reset or in a ready position or in an undeployed position.

3. The device of claim 2, wherein the knife safety lockout element is in the active state when the staple cartridge is removed from the staple holder or when the staple cartridge is empty or partially empty.

4. The device of claim 1, where the knife safety lockout element is in a neutral state when a new or fully loaded staple cartridge is placed into the staple holder.

5. The device of claim 2, wherein the knife safety lockout element is reset to a neutral state when a new or fully loaded staple cartridge is placed into the staple holder.
